(19) United States
(12) Patent Application Publication
(10) Pub. No.: US 2022/0010234 A1
(43) Pub. Date: Jan. 13, 2022

(54) ALKYLBENZENE SULFONATE DETERGENTS

(71) Applicant: The Lubrizol Corporation, Wickliffe, OH (US)

(72) Inventors: Ewan E. Delbridge, Concord Township, OH (US); James D. Burrington, Gates Mills, OH (US); Patrick E. Mosier, Bay Village, OH (US); James S. Puckace, Palm Harbor, FL (US)

(73) Assignee: The Lubrizol Corporation, Wickliffe, OH (US)

(21) Appl. No.: 17/293,836

(22) PCT Filed: Nov. 15, 2019
(86) PCT No.: PCT/US19/61708

§ 371 Date: May 13, 2021

Related U.S. Application Data
(60) Provisional application No. 62/768,219, filed on Nov. 16, 2018.

(51) Int. Cl. C10M 169/04; C10M 135/10; C10M 137/10; C10M 149/10; C10M 141/10; C10M 161/00; C07C 309/31

(52) U.S. Cl. CPC C10M 169/044; C10M 135/10; C10M 137/10; C10M 149/10; C10M 141/10; C10N 2030/04; C07C 309/31; C10M 2203/003; C10M 2219/046; C10M 2223/045; C10M 2217/028; C10M 161/00

(57) ABSTRACT

The instant disclosure relates to hydrocarbyl- (e.g., alkyl-) benzene sulfonate detergents and their salts, where the hydrocarbyl group includes moieties equivalent to 5 to 10 carbon branched polyene compounds. Such compounds and their salts are useful as lubricant additives. The detergents disclosed herein may solve at least one problem of providing anti-wear performance, frictional performance, oxidation performance, viscosity performance, and detergency.

### FIELD

The instant disclosure relates to hydrocarbyl- (e.g. alkyl-) benzene sulfonate detergents and their salts having oligomers of branched olefins, including branched polyenes, such as terpenes. Such compounds and their salts are useful as lubricant additives.

### BACKGROUND

Modern vehicles demand more efficiency and better emissions. As a result, there is an increasing focus on lubricants to provide improved fuel economy without sacrificing cleanliness or emissions quality. In addition, resourcing and sustainability continue to be major drivers of lubrication technology. Thus, there is a desire for improved detergents that provide the performance levels and may be sourced via more sustainable, e.g., biorenewable, processes.

### SUMMARY

The instant disclosure may solve at least one problem of providing a alkylbenzene sulfonate material or salt thereof with appropriate oil solubility, providing anti-wear performance, frictional performance, providing oxidation performance, viscosity performance, and detergency (characteristic of moderate chain length alkyl groups.

One aspect of the instant disclosure relates to an alkylbenzene sulfonate detergent composition comprising at least one benzene sulfonate moiety having a hydrocarbyl group attached thereto. The hydrocarbyl group can include at least one oligomer having monomers equivalent, in some embodiments, to 5 to 10 carbon atom branched olefins, and in some embodiments, more particularly to 5 to 10 carbon atom branched polyenes. In embodiments, the monomers making up the oligomers can be equivalent to isoprene. The oligomer itself can be equivalent to a terpene, and more particularly can be equivalent to any of (2E,5E,7E,10E)-3,6,10-trimethyldodeca-2,5,7,10-tetraene; (3E,7E,10E)-2,7,10-trimethyldodeca-1,3,7,10-tetraene; (E)-2,9-dimethyl-5-(prop-1-ene-2-yl)deca-1,3,8-triene; (3E,6E,10E)-2,6,10-trimethyldodeca-1,3,6,10-tetraene; (E)-7,11-dimethyl-3-methylenedodeca-1,6,10-triene; (6E,10E)-7,11,15-trimethyl-3-methylenehexadeca-1,6,10,14-tetraene; (3E,6E, 10E)-3,7,11,15-tetramethylhexadeca-1,3,6,10,1-pentaene; (6E,10E,14E)-7,11,14-trimethyl-3-methylenehexadeca-1,6,10,14-tetraene; (3E,9E,13E)-2, 10,13-trimethyl-6-(prop-1-en-2-yl)pentadeca-1,3,9,13-tetraene; and 3,7,11,15-tetramethylhexadec-1-ene.

The oligomer can also be equivalent to a hydrogenated (partial or complete) form of a terpene, such as, for example, any of 3,7,11-trimethyldodec-1-ene; 2,6,10-trimethyldodec-2-ene; (E)-3,7,11-trimethyldodec-2-ene; 2,6-dimethyl-10-methylenedodecane; (E)-2,6,10-trimethyldodec-6-ene; (E)-3,7,11-trimethyldodec-3-ene; (E)-2,6,10-trimethyldodec-5-ene; 3,7,11-trimethyldodeca-1,10-diene; and 3,7,11,15-tetrametylhexade-1-ene.

In some embodiments, the hydrocarbyl unit of the alkylbenzene sulfonate detergent can contain from 15 to 60 carbon atoms.

In an embodiment, the alkylbenzene sulfonate detergent can be an overbased detergent with a metal ratio of at least 1.5.

In an embodiment, the alkylbenzene sulfonate detergent can be a neutral or overbased salt.

Another embodiment relates to a lubricating composition having (a) an oil of lubricating viscosity and (b) the alkylbenzene sulfonate detergent composition as described herein. The lubricating composition can also include other additives, such as, for example, other detergents besides the instant alkylbenzene sulfonate detergent described herein, zinc dialkyldithiophosphate, dispersants, antiwear agents, extreme pressure agents, corrosion inhibitors, etc.

A further embodiment relates to methods of lubricating a mechanical device, for example, by supplying to the device the lubricating composition described herein containing the alkylbenzene sulfonate detergent. The mechanical device can be, for example, an internal combustion engine, an automotive driveline device, such as a transmission or an axle, or an off-highway vehicle. The technology could also be employed in industrial applications, such as industrial hydraulics.

### DETAILED DESCRIPTION

Various preferred features and embodiments will be described below by way of non-limiting illustration.

The instant disclosure includes an alkylbenzene sulfonate detergent composition, a lubricating composition containing the alkylbenzene sulfonate, a method for lubricating a mechanical device with a lubricating composition containing the alkylbenzene sulfonate, and a use of the alkylbenzene sulfonate detergent, all of which will be more particularly described herein. Reference to alkylbenzene sulfonate alone shall herein mean the disclosed alkylbenzene sulfonate detergent.

One embodiment relates to an alkylbenzene sulfonate detergent composition. The alkylbenzene sulfonate detergent will include an alkylbenzene sulfonate moiety with at least one hydrocarbyl group attached thereto. Those of ordinary skill in the art can observe a chemical composition and readily imagine the moieties to which the composition may be derived from. As used herein, reference to the benzene sulfonate moiety means that moiety of the alkylbenzene sulfonate detergent composition that one of ordinary skill in the art could ascertain would be derived from a benzene sulfonic acid or suitable derivative thereof, i.e., of Formula I.

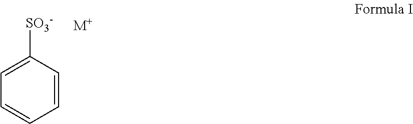

Where M is a H, ammonium, or a metal of proper valency. Examples of such metals include, without limitation, alkali metals, e.g. sodium and lithium; alkaline earth metals, e.g., magnesium, calcium; Group 3 through Group 12 transition metals, e.g. manganese, iron, copper, or zinc; and other main group metals, e.g. antimony.

The benzene sulfonate also includes a hydrocarbyl group. As used herein, the term “hydrocarbyl group” is used in its ordinary sense, which is well-known to those skilled in the art. Specifically, it refers to a group having a carbon atom directly attached to the remainder of the molecule and having predominantly hydrocarbon character. Examples of hydrocarbyl groups include, for example: hydrocarbon substituents, including aliphatic, alicyclic, and aromatic substituents; substituted hydrocarbon substituents, that is, substituents containing non-hydrocarbon groups which, in the context of this invention, do not alter the predominantly hydrocarbon nature of the substituent; and hetero substituents, that is, substituents which similarly have a predominantly hydrocarbon character but contain other than carbon in a ring or chain.

While the hydrocarbyl group can include elements of non-hydrocarbyl character (i.e., nitrogen, halogens, etc.), in many embodiments the hydrocarbyl group can be of substantially, or even completely, hydrocarbon character. In some embodiments, the hydrocarbyl group can be of substantially or completely aliphatic character.

The hydrocarbyl group can comprise, consist essentially of, or consist of at least one oligomer, and the at least one oligomer itself can comprise, consist essentially of, or consist of monomers equivalent to 5 to 10 carbon atom branched olefins. By employing the terminology “equivalent to,” it is recognized that the referenced compound, in this case the monomers of the oligomer, are slightly altered in their final state from their original state; for example, an oligomerized monomer versus the lone monomer. In other words, the hydrocarbyl group, when looked at in discreet units, contains identifiable oligomer units, and the identifiable oligomer units can be further broken down into identifiable alkyl units that as a monomer would be considered a 5 to 10 carbon atom branched olefin.

A branched olefin, as used herein, refers to an alkyl chain having at least one double bond and at least one tertiary carbon atom. Without limiting the branched olefin, examples of such branched olefins can be represented by the following exemplified branched olefin formulas.

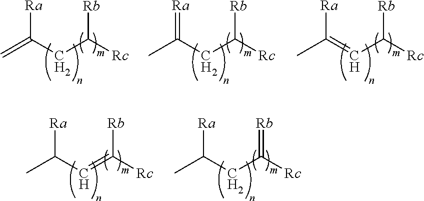

where Ra, Rb, and Rc can be H or a lower C₁ to C₅ alkyl group; n and m can be, independently, integers of 0 to 6, with the proviso that n+m is from 1 to 6; and the olefin has from 5 to 10 carbon atoms. An oligomer of such a branched olefin could be, for example, oligomers of the following formula.

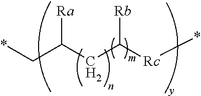

where Ra, Rb, Rc, m and n are as above, and y is an integer of 2 to 18, or 2 to 15, or 2 to 12.

The branched olefin can also be polyenes. That is, the hydrocarbyl group can comprise, consist essentially of, or consist of at least one oligomer, and the at least one oligomer itself can comprise, consist essentially of, or consist of monomers equivalent to 5 to 10 carbon atom branched polyene compounds. As used herein a polyene is a poly-unsaturated alkylene compound having at least 2 double bonds (also known as a diene), and in some cases 3, 4, or 5 double bonds.

Branched polyene compounds can be described as alkyl chains having at least two double bonds in the chain, and at least one tertiary carbon atom. Although the polyene compound may include more than 2 double bonds, an example branched polyene compound may be a branched conjugated diene represented by the following formula,

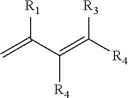

where R₁, R₂, R₃, and R₄ are independently H, or lower C₁ to C₅ alkyl groups; with the proviso that at least one of R₁, R₂, R₃, and R₄ is an alkyl group and that taken together the conjugated diene has a total of from 5 to 10 carbon atoms.

The at least one oligomer in the hydrocarbyl group can be oligomerized from 5 to 10 carbon atom branched olefin/polyenes, or the oligomer may be prepared in some other manner. For example, the oligomer may be formed by 1,2-monomer addition or 1,4-monomer addition. In one embodiment, oligomers of 5 to 10 carbon atom branched polyenes may formed by 1,4-addition of the monomers, followed by partial hydrogenation of the resulting material to form a mono-olefinic alkylating agent. In another example, the oligomer may also be produced via biological activity, such as through the fermentation of a cell culture system that is capable of producing the desired branched olefin/polyene or a composition having the appearance of an oligomer containing monomers equivalent to 5 to 10 carbon atom branched olefin/polyene compounds. For instance, WO 2011/160081 provides a biological pathway for the production of isoprene, and the use of the so-produced isoprene to produce oligomers (also called terpenes, i.e., having a formula of (C₅H₈)ₙ, where n is 2, 3, 4, 5 or higher). Other biological pathways are known to produce the oligomers (e.g., terpenes) themselves.

An oligomer containing monomers equivalent to 5 to 10 carbon atom branched olefins could be, for example, oligomers of the following formula

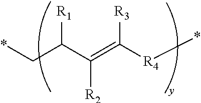

where R1, R2, R3, and R4 and y are the same as defined above.

The branched polyene compound containing 5 to 10 carbon atoms may include isoprene monomers of Formula IV.

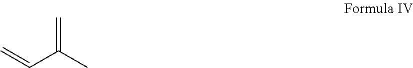

In addition to isoprene, suitable branched olefins 2-methyl-1-butene, 3-methyl-1-butene, 4-methyl-1-pentene, 2-methyl-1-pentene, 3-methyl-1-pentene, 2-methyl-1-hexene, 3-methyl-1-hexene, 5-methyl-1-hexene, 4-methyl-1-hexene, 2-methyl-1-heptene, 2.3-dimethyl-1,3-butadiene, 2,4-dimethyl-1,3-pentadiene, 3-methyl-1,3-pentadiene, and mixtures thereof.

In an embodiment, the oligomer contains units equivalent to isoprene. An isoprene oligomer containing 15 to 60 carbon atoms would contain 3 to 12 isoprene monomer units. An isoprene polymer or oligomer containing 15 to 30 carbon atoms would contain 3 or 6 isoprene monomer units.

In an embodiment, the oligomer can be a trimer of isoprene, which can be envisioned in the following forms

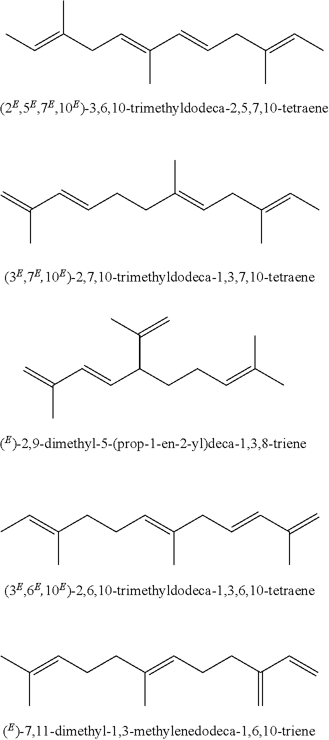

Tetramers of isoprene may also be employed in the oligomer, and can be envisioned in the following forms

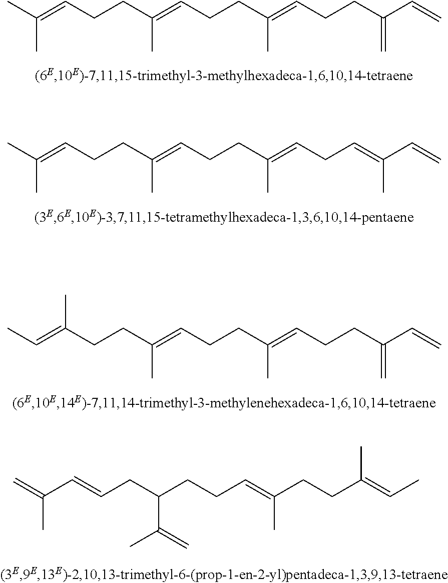

In one embodiment, the hydrocarbyl group of the alkyl benzene sulfonate detergent can comprise, consist essentially of, or consist of oligomer compounds comprising, consisting essentially of, or consisting of at least 50 mol % monomers equivalent to isoprene, at least 75 mol % monomers equivalent to isoprene, or at least 90 mol % monomers equivalent to isoprene. In one embodiment, the hydrocarbyl group in the alkyl benzene sulfonate detergent consists of oligomers of isoprene.

In some embodiments, the oligomer can be hydrogenated. Hydrogenation may be performed, for example, by any hydrogenating agent known to a skilled artisan. For example, a saturated oligomer of polyene compounds can be prepared by hydrogenating at least a portion of the double bonds in the oligomer in the presence of a hydrogenation reagent, such as hydrogen in the presence of a catalyst, or by treatment with hydrazine in the presence of a catalyst.

In some embodiments, at least a portion of the C═C bonds of the oligomer are reduced to the corresponding C—C bonds by hydrogenation. In some embodiments, all of the C═C bonds of the oligomer are reduced to the corresponding C—C bonds by hydrogenation. In an embodiment, the oligomer can include a hydrogenated form of oligomer as disclosed herein, including those in the form of any of the following structures:

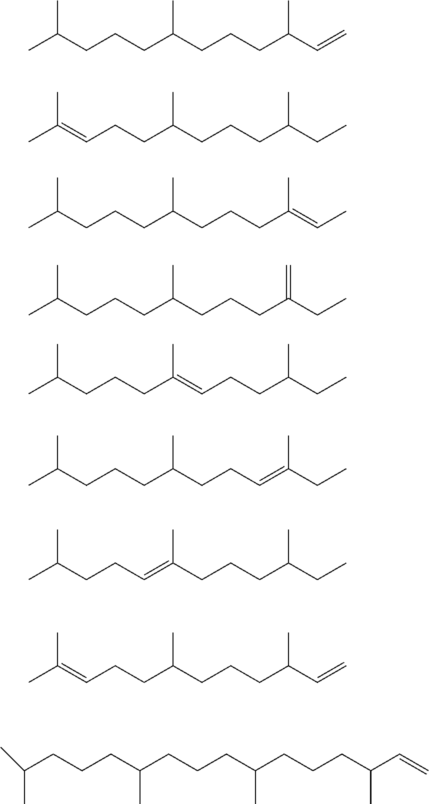

Whether prepared from the oligomerization of discreet units or otherwise, one of ordinary skill in the art will recognize the presence of the oligomers and monomers therein equivalent to 5 to 10 carbon atom branched polyenes within the hydrocarbyl group.

The hydrocarbyl group can contain from 10 to 200 carbon atoms, or in some examples 12 to 100 carbon atoms, or even 15 to 80 carbon atoms. In some embodiments, the hydrocarbyl group can have 15 to 60 carbon atoms, or in some cases 15 to 30 or 45 carbon atoms, or 20 to 30 carbon atoms.

The alkylbenzene sulfonate detergent, including the benzene sulfonate moiety and the hydrocarbyl group, may include the structure represented by the following formula.

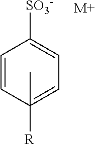

Where R represents the hydrocarbyl containing from 10 to 200 carbon atoms, or in some examples 12 to 100 carbon atoms, or even 15 to 80 carbon atoms and M is a H, ammonium, or a metal of proper valency. Examples of such metals include, without limitation, alkali metals, e.g., sodium and lithium; alkaline earth metals, e.g., magnesium, calcium; Group 3 through Group 12 transition metals, e.g., manganese, iron, copper, or zinc; and other main group metals, e.g., antimony. In some embodiments, the R hydrocarbyl group can have 15 to 60 carbon atoms, or in some cases 15 to 30 or 45 carbon atoms, or 20 to 30 carbon atoms. In some embodiments, the alkylbenzene sulfonate can include the structure shown in any one of the following formulas.

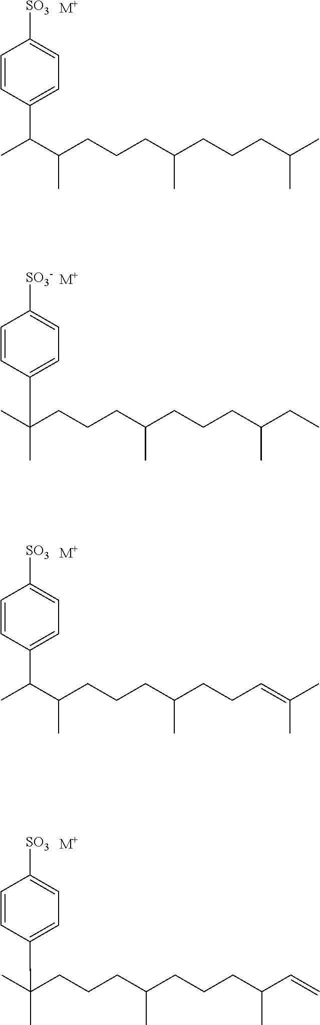

Where M is defined above. In one embodiment, M is hydrogen. In another embodiment, M is an alkaline earth metal including calcium, magnesium, or mixtures thereof.

In another embodiment, M is ammonium, quaternary ammonium, or mixtures thereof. Quaternary ammonium (nitrogen) compounds are known. Ordinarily nitrogen is a trivalent element, forming three covalent bonds to hydrogen or carbon atoms in ammonia or amines: NHₓR₃₋ₓ, where R is a group linked to the nitrogen atom through a carbon atom of the R group. Quaternary nitrogen compounds, on the other hand, comprise a quaternary ammonium ion and a counterion (e.g., hydroxide, halide), represented by the general formula:

NR₄⁺X⁻

where, each R independently represents a suitable hydrocarbyl group, and X⁻ represents the sulfonate of the invention and may include one equivalent of an anionic counterion, which may include fractional equivalents of polyanionic species (e.g. a half mole of carbonate, i.e., ½CO²⁻).

In one embodiment, the alkyl group (or alkylate), i.e., the R group, of the alkylbenzene sulfonate detergent comprises at least 25 wt %, or at least 50 wt %, or at least 75 wt %, or at least 80 wt % or at least 90 wt %, or at least 95 wt % of biorenewable content. In another embodiment, the alkyl group (or alkylate) comprises 100 wt % of biorenewable content. “Biorenewable” means that the described biorenwable element is generated from a feedstock created by biological organisms, as described herein.

The alkylbenzene sulfonate detergents described herein may be a metal-containing detergent. Metal-containing detergents may be neutral, or very nearly neutral, or overbased. An overbased detergent contains a stoichiometric excess of a metal base for the acidic organic substrate. This is also referred to as metal ratio. The term “metal ratio” is the ratio of the total equivalents of the metal (or equivalent counter cation, such as ammonium) to the equivalents of the acidic organic compound. A neutral metal salt has a metal ratio of one. A salt having 4.5 times as much metal as present in a normal salt will have metal excess of 3.5 equivalents, or a ratio of 4.5. The term “metal ratio is also explained in standard textbook entitled “Chemistry and Technology of Lubricants”, Third Edition, Edited by R. M. Mortier and S. T. Orszulik, Copyright 2010, page 219, sub-heading 7.25.

Alternatively, the alkylbenzene sulfonate detergent may be described as having total base number (“TBN”), calculated on an oil-free basis. In some embodiments, the TBN can range from 25 to 800 mg KOH/g, or 115 to 650 mg KOH/g, or 180 to 550 mg KOH/g. In some embodiments, the TBN is at least 400. In another embodiment, the TBN is at least 425. In yet another embodiment, the TBN is at least 450. In one embodiment, the TBN is at least 490. In one embodiment, an overbased alkylbenzene sulfonate detergent has a TBN of 480 to 700. The amount of the alkylbenzene sulfonate detergent present in a lubricant composition may be defined as the amount necessary to deliver an amount, or range of amounts, of TBN to the lubricant composition. In certain embodiments, the alkylbenzene sulfonate detergent may be present in a lubricant composition in amount to deliver 0.5 to 10 TBN to the composition, or 1 to 7 TBN, or 1.5 to 5 TBN to the composition.

Overbased detergents may also be defined as the ratio of the neutral detergent salt, also referred to as detergent soap, and the detergent ash. The overbased detergent may have a weight ratio of ash to soap of 3:1 to 1:8, or 1.5:1 to 1 to 4.1, or 1.3:1 to 1:3.4.

The metal compounds useful in making the basic metal salts of the alkylbenzene sulfonate detergent are generally any Group 1 or Group 2 metal compounds (CAS version of the Periodic Table of the Elements). The Group 1 metals of the metal compound include Group la alkali metals such as sodium, potassium, and lithium, as well as Group 1b metals such as copper. The Group 1 metals can be sodium, potassium, lithium and copper, and in one embodiment sodium or potassium, and in another embodiment, sodium. The Group 2 metals of the metal base include the Group 2a alkaline earth metals such as magnesium, calcium, and barium, as well as the Group 2b metals such as zinc or cadmium. In one embodiment, the Group 2 metals are magnesium, calcium, barium, or zinc, and in another embodiment magnesium or calcium. In certain embodiments, the metal is calcium or sodium or a mixture of calcium and sodium. Generally, the metal compounds are delivered as metal salts. The anionic portion of the salt can be hydroxide, oxide, carbonate, borate, or nitrate. Commonly used basic metal compounds include calcium oxide and calcium hydroxide.

The overbasing may include mixing of the neutral alkylbenzene sulfonate salt with a stoichiometric excess of basic compound (e.g., a basic metal compound such as calcium oxide or calcium hydroxide or alternatively compounds such as, magnesium hydroxide, magnesium oxide, sodium hydroxide, or sodium oxide; or alternatively a basic nitrogen compound such as ammonia or an amine), typically in a solvent such as mineral oil, and typically in the presence of one or more promoters such as alcohols. Typical alcoholic promoters include mixtures of methanol, isobutyl alcohol, and/or amyl alcohols, in various proportions. Optionally, a small amount of a dispersant or another detergent may be present. The mixture is treated with an acidic gas, such as typically CO₂, which will convert at least a portion of the excess basic compound to the salt, such as CₐCO₃. The addition of the basic compound and the subsequent treatment with the acidic gas may be conducted in several portions or iterations, which may permit formation of materials with a higher metal ratio and total base number. After the reaction is complete, volatile components may be removed.

In one embodiment, the alkylbenzene sulfonate detergent may comprise a calcium or magnesium alkylbenzene sulfonate detergent with a metal ratio of 1 to 40. In another embodiment, the metal ratio is 1 to 3, 3 to 30, or 4 to 25, or 5 to 20, or 6 to 15, or 8 to 12, or 8 to 24. In other embodiments, the metal ratio is at least 1, at least 3, at least 5, or at least 7, or at least 10. In certain embodiments, the overbased calcium alkylbenzene sulfonate detergent may have a metal ratio of 1.5 to 25, 2.5 to 20 or 5 to 16.

In one embodiment, the overbased alkylbenzene sulfonate detergent can be a neutral detergent or substantially neutral detergent. In some embodiments, the overbased alkylbenzene sulfonate detergent has a TBN of 0 to 120. In another embodiment, the overbased alkylbenzene detergent has a TBN of 5 to 80. In yet another embodiment, the overbased alkylbenzene detergent has a TBN of 10 to 40.

The overbased alkylbenzene sulfonate detergent may be used as an additive in a lubricant. The amount of the alkylbenzene sulfonate detergent in a lubricant may be 0.1 to 8 percent by weight, on an oil-free basis, but including the calcium carbonate and other salts present in an overbased composition. In marine diesel lubricant compositions, the overbased alkylbenzene sulfonate detergent may be present in an amount of from 1 to 25, or 5 to 15, or 10 to 20 percent by weight. Amounts used in lubricants for gasoline or heavy-duty diesel engines (not marine) may be used in amounts ranging from 0.1 to 10 percent or 0.5 to 5 or 1 to 3 percent by weight.

In certain embodiments, the amount of the alkylbenzene sulfonate detergent in a lubricant may be measured as the amount of alkylbenzene sulfonate-containing soap that is provided to the lubricant composition, irrespective of any overbasing. In one embodiment, the lubricant composition may contain 0.05 weight percent to 1.5 weight percent alkylbenzene sulfonate-containing soap, or 0.1 weight percent to 0.9 weight percent alkylbenzene sulfonate-containing soap. In one embodiment, the alkylbenzene sulfonate-containing soap provides at least 20 weight percent, or at least 40 weight percent, or at least 70 weight percent, or at least 90 weight percent of the total detergent soap in the lubricating composition. In one embodiment, the alkylbenzene sulfonate-containing soap provides 20 percent by weight to 100 percent by weight of the total detergent soap in the lubricating composition. In one embodiment the alkylbenzene sulfonate-containing soap provides 30 percent by weight to 80 percent by weight of the total detergent soap, or 40 percent by weight to 75 percent by weight of the total detergent soap of the lubricating composition.

A lubricant composition may contain alkyl sulfonate-containing detergents different from the alkylbenzene sulfonate detergent disclosed herein. In one embodiment, a lubricant composition may comprise the instantly disclosed alkylbenzene sulfonate detergent in an amount of from 0.1 to 25 weight percent, or 0.2 to 23, or 0.3 to 20, or 0.5 to 15 weight percent, and is free of or substantially free of other alkyl sulfonate-containing detergents. “Substantially free of” in this case means no more than 0.01 weight percent or an amount considered to arise through contamination or other unintentional means. In some embodiments, the lubricant composition can include the alkyl sulfonate-containing detergent at from about 0.01 to about 2 wt %, or from about 0.1 to about 1.75 wt %, or about 0.2 to about 1.5 wt % of the lubricating composition.

### Oil of Lubricating Viscosity

Another aspect of the technology is a lubricant containing the alkyl benzene sulfonate detergent composition. The lubricating composition includes an oil of lubricating viscosity. Such oils include natural and synthetic oils, oil derived from hydrocracking, hydrogenation, and hydro-finishing, unrefined, refined, re-refined oils or mixtures thereof. A more detailed description of unrefined, refined and re-refined oils is provided in International Publication WO2008/147704, paragraphs [0054] to [0056] (a similar disclosure is provided in US Patent Application 2010/197536, see [0072] to [0073]). A more detailed description of natural and synthetic lubricating oils is described in paragraphs [0058] to [0059] respectively of WO2008/147704 (a similar disclosure is provided in US Patent Application 2010/197536, see [0075] to [0076]). Synthetic oils may also be produced by Fischer-Tropsch reactions and typically may be hydroisomerized Fischer-Tropsch hydrocarbons or waxes. In one embodiment oils may be prepared by a Fischer-Tropsch gas-to-liquid synthetic procedure as well as other gas-to-liquid oils.

Oils of lubricating viscosity may also be defined as specified in the American Petroleum Institute (API) Base Oil Interchangeability Guidelines (2011). The five base oil groups are as follows: Group I (sulfur content>0.03 wt %, and/or <90 wt % saturates, viscosity index 80 to less than 120); Group II (sulfur content ≤0.03 wt %, and ≥90 wt % saturates, viscosity index 80 to less than 120); Group III (sulfur content ≤0.03 wt %, and ≥90 wt % saturates, viscosity index ≥120); Group IV (all polyalphaolefins (PAOs)); and Group V (all others not included in Groups I, II, III, or IV). The oil of lubricating viscosity may also be a Group II+ base oil, which is an unofficial API category that refers to a Group II base oil having a viscosity index greater than or equal to 110 and less than 120, as described in SAE publication “Design Practice: Passenger Car Automatic Transmissions,” fourth Edition, AE-29, 2012, page 12-9, as well as in U.S. Pat. No. 8,216,448, column 1 line 57. The oil of lubricating viscosity may also be a Group III+ base oil, which, again, is an unofficial API category that refers to a Group III base oil having a viscosity index of greater than 130, for example 130 to 133 or even greater than 135, such as 135-145. Gas to liquid (“GTL”) oils are sometimes considered Group III+ base oils.

The oil of lubricating viscosity may be an API Group IV oil, or mixtures thereof, i.e., a polyalphaolefin. The polyalphaolefin may be prepared by metallocene catalyzed processes or from a non-metallocene process. The oil of lubricating viscosity may also comprise an API Group I, Group II, Group III, Group IV, Group V oil or mixtures thereof. Often the oil of lubricating viscosity is an API Group I, Group II, Group II+, Group III, Group IV oil or mixtures thereof. Alternatively, the oil of lubricating viscosity is often an API Group II, Group II+, Group III or Group IV oil or mixtures thereof. Alternatively, the oil of lubricating viscosity is often an API Group II, Group II+, Group III oil or mixtures thereof.

The oil of lubricating viscosity, or base oil, will overall have a kinematic viscosity at 100° C. of 2 to 10 cSt or, in some embodiments 2.25 to 9 or 2.5 to 6 or 7 or 8 cSt, as measured by ASTM D445. Kinematic viscosities for the base oil at 100° C. of from about 3.5 to 6 or from 6 to 8 cSt are also suitable.

The amount of the oil of lubricating viscosity present is typically the balance remaining after subtracting from 100 wt % the sum of the amount of the performance additives in the composition. Illustrative amounts may include 50 to 99 percent by weight, or 60 to 98, or 70 to 95, or 80 to 94, or 85 to 93 percent.

The lubricating composition may be in the form of a concentrate and/or a fully formulated lubricant. If the lubricating composition of the invention is in the form of a concentrate (which may be combined with additional oil to form, in whole or in part, a finished lubricant), the ratio of the of components of the invention to the oil of lubricating viscosity and/or to diluent oil include the ranges of 1:99 to 99:1 by weight, or 80:20 to 10:90 by weight.

A lubricating composition may be prepared by adding the product of the process described herein to an oil of lubricating viscosity, optionally in the presence of other performance additives (as described herein below).

### Other Performance Additives

The other performance additives can include at least one of metal deactivators, viscosity modifiers, detergents (other than the alkylbenzene sulfonate disclosed herein), friction modifiers, antiwear agents, corrosion inhibitors, dispersants, extreme pressure agents, antioxidants, foam inhibitors, demulsifiers, pour point depressants, seal swelling agents and mixtures thereof. Typically, fully-formulated lubricating oil will contain one or more of these performance additives.

In one embodiment, the invention provides a lubricating composition further comprising an overbased metal-containing detergent in addition to the alkyl benzene sulfonate-containing detergent of the present invention. The metal of the metal-containing detergent may be zinc, sodium, calcium, barium, or magnesium. Typically, the metal of the metal-containing detergent may be sodium, calcium, or magnesium.

The overbased metal-containing detergent may be chosen from sulfonates, non-sulfur containing phenates, sulfur containing phenates, salixarates, salicylates, and mixtures thereof, or borated equivalents thereof. The overbased detergent may be borated with a borating agent such as boric acid.

The overbased metal-containing detergent may also include “hybrid” detergents formed with mixed surfactant systems including phenate and/or sulfonate components, e.g., phenate/salicylates, sulfonate/phenates, sulfonate/salicylates, sulfonates/phenates/salicylates, as described; for example, in U.S. Pat. Nos. 6,429,178; 6,429,179; 6,153,565; and 6,281,179. Where, for example, a “hybrid” sulfonate/phenate detergent is employed, the “hybrid” detergent would be considered equivalent to amounts of distinct phenate and sulfonate detergents introducing like amounts of phenate and sulfonate soaps, respectively.

Typically, an overbased metal-containing detergent may be a zinc, sodium, calcium or magnesium salt of a sulfonate, a phenate, sulfur containing phenate, salixarate or salicylate. Overbased sulfonates, salixarates, phenates and salicylates typically have a total base number of 120 to 700 TBN.

Typically, the overbased metal-containing detergent may be a calcium or magnesium overbased detergent.

In another embodiment, the lubricating composition further comprises a calcium sulfonate overbased detergent having a TBN of 120 to 700. The overbased sulfonate detergent may have a metal ratio of 12 to less than 20, or 12 to 18, or 20 to 30, or 22 to 25.

Overbased sulfonates typically have a total base number of 120 to 700, or 250 to 600, or 300 to 500 (on an oil free basis). Overbased detergents are known in the art. In one embodiment the sulfonate detergent may be a predominantly linear alkylbenzene sulfonate detergent having a metal ratio of at least 8 as is described in paragraphs [0026] to [0037] of US Patent Application 2005065045 (and granted as U.S. Pat. No. 7,407,919). Linear alkyl benzenes may have the benzene ring attached anywhere on the linear chain, usually at the 2, 3, or 4 position, or mixtures thereof. The predominantly linear alkylbenzene sulfonate detergent may be particularly useful for assisting in improving fuel economy. In one embodiment, the sulfonate detergent may be a metal salt of one or more oil-soluble alkyl toluene sulfonate compounds as disclosed in paragraphs [0046] to [0053] of US Patent Application 2008/0119378.

In one embodiment, the lubricating composition further comprises 0.01 wt % to 2 wt %, or 0.1 to 1 wt % of a detergent different from the alkyl benzene sulfonate detergent of the disclosed technology, wherein the further detergent is chosen from sulfonates, non-sulfur containing phenates, sulfur containing phenates, sulfonates, salixarates, salicylates, and mixtures thereof, or borated equivalents thereof.

In one embodiment, the lubricating composition does not include, i.e. contains 0 weight percent, an alkylbenzene sulfonate detergent different from that of the alkylbenzene sulfonate detergent disclosed herein.

In one embodiment, the lubricating composition further comprises a “hybrid” detergent formed with mixed surfactant systems including phenate and/or sulfonate components, e.g. phenate/salicylates, sulfonate/phenates, sulfonate/salicylates, or sulfonates/phenates/salicylates.

The lubricating composition in a further embodiment comprises an antioxidant, wherein the antioxidant comprises a phenolic or an aminic antioxidant or mixtures thereof. The antioxidants include diarylamines, alkylated diarylamines, hindered phenols, or mixtures thereof. When present the antioxidant is present at 0.1 wt % to 3 wt %, or 0.5 wt % to 2.75 wt %, or 1 wt % to 2.5 wt % of the lubricating composition.

The diarylamine or alkylated diarylamine may be a phenyl-α-naphthylamine (PANA), an alkylated diphenylamine, or an alkylated phenylnapthylamine, or mixtures thereof. The alkylated diphenylamine may include di-nonylated diphenylamine, nonyl diphenylamine, octyl diphenylamine, di-octylated diphenylamine, di-decylated diphenylamine, decyl diphenylamine and mixtures thereof. In one embodiment the diphenylamine may include nonyl diphenylamine, dinonyl diphenylamine, octyl diphenylamine, dioctyl diphenylamine, or mixtures thereof. In another embodiment, the alkylated diphenylamine may include nonyl diphenylamine, or dinonyl diphenylamine. The alkylated diarylamine may include octyl, di-octyl, nonyl, di-nonyl, decyl or di-decyl phenylnapthylamines.

The hindered phenol antioxidant often contains a secondary butyl and/or a tertiary butyl group as a sterically hindering group. The phenol group may be further substituted with a hydrocarbyl group (typically linear or branched alkyl) and/or a bridging group linking to a second aromatic group. Examples of suitable hindered phenol antioxidants include 2,6-di-tert-butylphenol, 4-methyl-2,6-di-tert-butylphenol, 4-ethyl-2,6-di-tert-butylphenol, 4-propyl-2,6-di-tert-butyl¬phenol or 4-butyl-2,6-di-tert-butylphenol, or 4-dodecyl-2,6-di-tert-butyl¬phenol. In one embodiment, the hindered phenol antioxidant may be an ester and may include, e.g., Irganox™ L-135 from Ciba. A more detailed description of suitable ester-containing hindered phenol antioxidant chemistry is found in U.S. Pat. No. 6,559,105.

The lubricating composition may in a further embodiment include a dispersant, or mixtures thereof. The dispersant may be a succinimide dispersant, a Mannich dispersant, a succinamide dispersant, a polyolefin succinic acid ester, amide, or ester-amide, or mixtures thereof. In one embodiment, the dispersant may be present as a single dispersant. In one embodiment, the dispersant may be present as a mixture of two or three different dispersants, wherein at least one may be a succinimide dispersant.

The succinimide dispersant may be derived from an aliphatic polyamine, or mixtures thereof. The aliphatic polyamine may be aliphatic polyamine such as an ethylenepolyamine, a propylenepolyamine, a butylenepolyamine, or mixtures thereof. In one embodiment, the aliphatic polyamine may be ethylenepolyamine. In one embodiment, the aliphatic polyamine may be chosen from ethylenediamine, diethylenetriamine, triethylenetetramine, tetra¬ethylene¬pentamine, pentaethylene-hexamine, polyamine still bottoms, and mixtures thereof.

In one embodiment, the dispersant may be derived from an aliphatic polyether, an aliphatic polyether amine, an aliphatic polyether polyamine, or mixtures thereof. Typical polyether compounds contain at least one ether unit and will be chain terminated with at least one hydroxy or amine moiety. Polyethers can be based on polymers derived from C₂-C₆ epoxides such as ethylene oxide, propylene oxide, and butylene oxide. Examples of polyether polyamines are sold under the Jeffamine® brand and are commercially available from Hunstman Corporation located in Houston, Tex.

In one embodiment, the dispersant may be a polyolefin succinic acid ester, amide, or ester-amide. For instance, a polyolefin succinic acid ester may be a polyisobutylene succinic acid ester of pentaerythritol, or mixtures thereof. A polyolefin succinic acid ester-amide may be a polyisobutylene succinic acid reacted with an alcohol (such as pentaerythritol) and a polyamine as described above.

The dispersant may be an N-substituted long chain alkenyl succinimide. An example of an N substituted long chain alkenyl succinimide is polyisobutylene succinimide. Typically, the polyisobutylene from which polyisobutylene succinic anhydride is derived has a number average molecular weight of 350 to 5000, or 550 to 3000 or 750 to 2500. Succinimide dispersants and their preparation are disclosed, for instance in U.S. Pat. Nos. 3,172,892, 3,219,666, 3,316,177, 3,340,281, 3,351,552, 3,381,022, 3,433,744, 3,444,170, 3,467,668, 3,501,405, 3,542,680, 3,576,743, 3,632,511, 4,234,435, Re 26,433, and 6,165,235, 7,238,650 and EP Patent Application 0 355 895 A.

The dispersants may also be post-treated by conventional methods by a reaction with any of a variety of agents. Among these are boron compounds (such as boric acid), carbonate compounds (such as ethylene carbonate), urea, thiourea, dimercaptothiadiazoles, carbon disulfide, aldehydes, ketones, carboxylic acids such as terephthalic acid, hydrocarbon-substituted succinic anhydrides, maleic anhydride, nitriles, epoxides, and phosphorus compounds. In one embodiment, the post-treated dispersant is borated. In one embodiment, the post-treated dispersant is reacted with dimercaptothiadiazoles. In one embodiment, the post-treated dispersant is reacted with phosphoric or phosphorous acid. In one embodiment, the post-treated dispersant is reacted with terephthalic acid and boric acid (as described in US Patent Application US2009/0054278.

When present, the dispersant may be present at 0.01 wt % to 20 wt %, or 0.1 wt % to 15 wt %, or 0.1 wt % to 10 wt %, or 1 wt % to 6 wt %, or 1 to 3 wt %, or 2 wt % to 4 wt % of the lubricating composition.

In one embodiment, the lubricating composition further comprises an ashless friction modifier. Suitable friction modifiers may be chosen from long chain fatty acid derivatives of amines, long chain fatty esters, or derivatives of long chain fatty epoxides; fatty imidazolines; amine salts of alkylphosphoric acids; fatty alkyl tartrates; fatty alkyl tartrimides; fatty alkyl tartramides; fatty glycolates; and fatty glycolamides. The friction modifier may be present at 0 wt % to 6 wt %, or 0.01 wt % to 4 wt %, or 0.05 wt % to 2 wt %, or 0.1 wt % to 2 wt %, or 0.15 wt % to 1.5 wt % of the lubricating composition.

As used herein the term “fatty alkyl” or “fatty” in relation to friction modifiers means a carbon chain having 10 to 22 carbon atoms, typically a straight carbon chain.

Examples of suitable friction modifiers include long chain fatty acid derivatives of amines, fatty esters, or fatty epoxides; fatty imidazolines such as condensation products of carboxylic acids and polyalkylene-polyamines; amine salts of alkylphosphoric acids; fatty alkyl tartrates; fatty alkyl tartrimides; fatty alkyl tartramides; fatty phosphonates; fatty phosphites; borated phospholipids, borated fatty epoxides; glycerol esters; borated glycerol esters; fatty amines; alkoxylated fatty amines; borated alkoxylated fatty amines; hydroxyl and polyhydroxy fatty amines including tertiary hydroxy fatty amines; hydroxy alkyl amides; metal salts of fatty acids; metal salts of alkyl salicylates; fatty oxazolines; fatty ethoxylated alcohols; condensation products of carboxylic acids and polyalkylene polyamines; or reaction products from fatty carboxylic acids with guanidine, aminoguanidine, urea, or thiourea and salts thereof.

Friction modifiers may also encompass materials such as sulfurised fatty compounds and olefins, molybdenum dialkyldithiophosphates, molybdenum dithiocarbamates, sunflower oil or soybean oil monoester of a polyol and an aliphatic carboxylic acid.

In another embodiment, the friction modifier may be a long chain fatty acid ester. In another embodiment, the long chain fatty acid ester may be a mono-ester and in another embodiment the long chain fatty acid ester may be a triglyceride. In one embodiment, the friction modifier may be a mono-ester of glycerol (such as glycerol mono-oleate).

The lubricating composition optionally further includes at least one antiwear agent. Examples of suitable antiwear agents include titanium compounds, tartrates, tartrimides, oil soluble amine salts of phosphorus compounds, sulfurized olefins, metal dihydrocarbyldithiophosphates (such as zinc dialkyldithiophosphates), phosphites (such as dibutyl phosphite), phosphonates, thiocarbamate-containing compounds, such as thiocarbamate esters, thiocarbamate amides, thiocarbamic ethers, alkylene-coupled thiocarbamates, and bis(S-alkyldithiocarbamyl) disulfides.

The antiwear agent may in one embodiment include a tartrate, or tartrimide as disclosed in International Publication WO 2006/044411 or Canadian Patent CA 1 183 125. The tartrate or tartrimide may contain alkyl-ester groups, where the sum of carbon atoms on the alkyl groups is at least 8. The antiwear agent may in one embodiment include a citrate as is disclosed in US Patent Application 20050198894.

Another class of additives includes oil-soluble titanium compounds as disclosed in U.S. Pat. No. 7,727,943 and US2006/0014651. The oil-soluble titanium compounds may function as antiwear agents, friction modifiers, antioxidants, deposit control additives, or more than one of these functions. In one embodiment, the oil soluble titanium compound is a titanium (IV) alkoxide. The titanium alkoxide is formed from a monohydric alcohol, a polyol or mixtures thereof. The monohydric alkoxides may have 2 to 16, or 3 to 10 carbon atoms. In one embodiment, the titanium alkoxide is titanium (IV) isopropoxide. In one embodiment, the titanium alkoxide is titanium (IV) 2 ethylhexoxide. In one embodiment, the titanium compound comprises the alkoxide of a vicinal 1,2-diol or polyol. In one embodiment, the 1,2-vicinal diol comprises a fatty acid mono-ester of glycerol, often the fatty acid is oleic acid.

In one embodiment, the oil soluble titanium compound is a titanium carboxylate. In a further embodiment the titanium (IV) carboxylate is titanium neodecanoate.

The lubricating composition may in one embodiment further include a phosphorus-containing antiwear agent. Typically, the phosphorus-containing antiwear agent may be a zinc dialkyldithiophosphate, phosphite, phosphate, phosphonate, and ammonium phosphate salts, or mixtures thereof. Zinc dialkyldithiophosphates (ZDDP or ZDP) are known in the art. ZDDP's may be derived from primary aliphatic alcohols, secondary aliphatic alcohols, aromatic alcohols, and mixtures thereof. In one embodiment, the lubricating composition further comprises a ZDDP antiwear agent derived from at least 25 wt %, or at least 50 wt %, or at least 75 wt % secondary aliphatic alcohol. The antiwear agent may be present at 0 wt % to 3 wt %, or 0.1 wt % to 1.5 wt %, or 0.5 wt % to 0.9 wt % of the lubricating composition. In some embodiments, the antiwear agent may be present in an amount sufficient to provide from 0 to 0.12 wt % phosphorus to the lubricating composition, or from 0.01 to 0.08 wt %, or 0.03 to 0.08 wt %, or even 0.025 to 0.06 wt % phosphorus.

Extreme Pressure (EP) agents that are soluble in the oil include sulfur- and chlorosulfur-containing EP agents, dimercaptothiadiazole or CS₂ derivatives of dispersants (typically succinimide dispersants), derivative of chlorinated hydrocarbon EP agents and phosphorus EP agents. Examples of such EP agents include chlorinated wax; sulfurized olefins (such as sulfurized isobutylene), a hydrocarbyl-substituted 2,5-dimercapto-1,3,4-thiadiazole, or oligomers thereof, organic sulfides and polysulfides such as dibenzyldisulfide, bis-(chlorobenzyl) disulfide, dibutyl tetrasulfide, sulfurized methyl ester of oleic acid, sulfurized alkylphenol, sulfurized dipentene, sulfurized terpene, and sulfurized Diels-Alder adducts; phosphosulfurized hydrocarbons such as the reaction product of phosphorus sulfide with turpentine or methyl oleate; phosphorus esters such as the dihydrocarbon and trihydrocarbon phosphites, e.g., dibutyl phosphite, diheptyl phosphite, dicyclohexyl phosphite, pentylphenyl phosphite; dipentylphenyl phosphite, tridecyl phosphite, distearyl phosphite and polypropylene substituted phenol phosphite; metal thiocarbamates such as zinc dioctyldithiocarbamate and barium heptylphenol diacid; amine salts of alkyl and dialkylphosphoric acids or derivatives including, for example, the amine salt of a reaction product of a dialkyldithiophosphoric acid with propylene oxide and subsequently followed by a further reaction with P₂O₅; and mixtures thereof (as described in U.S. Pat. No. 3,197,405).

Foam inhibitors that may be useful in the lubricant compositions of the disclosed technology include polysiloxanes, copolymers of ethyl acrylate and 2-ethylhexylacrylate and optionally vinyl acetate; demulsifiers including fluorinated polysiloxanes, trialkyl phosphates, polyethylene glycols, polyethylene oxides, polypropylene oxides and (ethylene oxide-propylene oxide) polymers.

The lubricating composition may in one embodiment further include a viscosity modifier (VM), dispersant viscosity modifiers (DVM), or mixtures thereof. Viscosity modifiers, also called viscosity index improvers, are well known in the art. Viscosity modifiers include polymeric viscosity modifiers. The dispersant viscosity modifier may be generally understood to be a functionalized, i.e., derivatized, form of a polymer similar to that of the polymeric viscosity modifier.

The polymeric viscosity modifier may be an olefin (co)polymer (OCP), a poly(meth)acrylate (PMA), or mixtures thereof. In one embodiment, the polymeric viscosity modifier is an olefin (co)polymer.

The olefin polymer may be derived from isobutylene or isoprene. In one embodiment, the olefin polymer is prepared from ethylene and a higher olefin within the range of C3-C10 alpha-mono-olefins, for example, the olefin polymer may be prepared from ethylene and propylene.

In one embodiment, the olefin polymer may be a polymer of 15 to 80 mole percent of ethylene, for example, 30 mol percent to 70 mol percent ethylene and from and from 20 to 85 mole percent of C3 to C10 mono-olefins, such as propylene, for example, 30 to 70 mol percent propylene or higher mono-olefins. Terpolymer variations of the olefin copolymer may also be used and may contain up to 15 mol percent of a non-conjugated diene or triene. Non-conjugated dienes or trienes may have 5 to about 14 carbon atoms. The non-conjugated diene or triene monomers may be characterized by the presence of a vinyl group in the structure and can include cyclic and bicycle compounds. Representative dienes include 1,4-hexadiene, 1,4-cyclohexadiene, dicyclopentadiene, 5-ethyldiene-2-norbornene, 5-methylene-2-norbornene, 1,5-heptadiene, and 1,6-octadiene.

Useful olefin polymers, in particular, ethylene-α-olefin copolymers have a number average molecular weight ranging from 4500 to 500,000, for example, 5000 to 100,000, or 7500 to 60,000, or 8000 to 45,000.

In one embodiment, the DVM comprises a functionalized ethylene-α-olefin copolymer grafted with the acyl group which is further functionalized with a hydrocarbyl amine, a hydrocarbyl alcohol group, amino- or hydroxy-terminated polyether compounds, and mixtures thereof.

In one embodiment, lubricating composition may comprise a poly(meth)acrylate polymeric viscosity modifier. As used herein, the term “(meth)acrylate” and its cognates means either methacrylate or acrylate, as will be readily understood.

In one embodiment, the poly(meth)acrylate polymer is prepared from a monomer mixture comprising (meth)acrylate monomers having alkyl groups of varying length. The (meth)acrylate monomers may contain alkyl groups that are straight chain or branched chain groups. The alkyl groups may contain 1 to 24 carbon atoms, for example 1 to 20 carbon atoms.

In one embodiment, the poly(meth)acrylate polymers may have an architecture selected from linear, branched, hyper-branched, cross-linked, star (also referred to as “radial”), or combinations thereof. Star or radial refers to multi-armed polymers. Such polymers include (meth)acrylate-containing polymers comprising 3 or more arms or branches, which, in some embodiments, contain at least about 20, or at least 50 or 100 or 200 or 350 or 500 or 1000 carbon atoms. The arms are generally attached to a multivalent organic moiety which acts as a “core” or “coupling agent.” The multi-armed polymer may be referred to as a radial or star polymer, or even a “comb” polymer, or a polymer otherwise having multiple arms or branches as described herein.

Linear poly(meth)acrylates, random, block or otherwise, may have weight average molecular weight (M_{w}) of 1000 to 400,000 Daltons, 1000 to 150,000 Daltons, or 15,000 to 100,000 Daltons. In one embodiment, the poly(meth)acrylate may be a linear block copolymer with a Mw of 5,000 to 40,000 Daltons, or 10,000 to 30,000 Daltons.

Radial, cross-linked or star copolymers may be derived from linear random or di-block copolymers with molecular weights as described above. A star polymer may have a weight average molecular weight of 10,000 to 1,500,000 Daltons, or 40,000 to 1,000,000 Daltons, or 300,000 to 850,000 Daltons.

Other viscosity modifiers may include a block copolymer comprising (i) a vinyl aromatic monomer block and (ii) a conjugated diene olefin monomer block (such as a hydrogenated styrene-butadiene copolymer or a hydrogenated styrene-isoprene copolymer), or mixtures thereof.

The lubricating compositions may comprise 0.05 weight % to 10 weight %, or 0.3 weight % to 8 weight %, or 0.5 to 5 weight %, or 1 weight % to 3 weight % of the one or more polymeric viscosity modifiers and/or dispersant viscosity modifiers as described herein.

Pour point depressants that may be useful in the lubricant compositions of the disclosed technology include polyalphaolefins, esters of maleic anhydride-styrene copolymers, poly(meth)acrylates, polyacrylates or polyacrylamides.

Demulsifiers include trialkyl phosphates, and various polymers and copolymers of ethylene glycol, ethylene oxide, propylene oxide, or mixtures thereof.

Metal deactivators include derivatives of benzotriazoles (typically tolyltriazole), 1,2,4-triazoles, benzimidazoles, 2-alkyldithiobenzimidazoles or 2-alkyldithiobenzothiazoles. The metal deactivators may also be described as corrosion inhibitors.

Seal swell agents include sulfolene derivatives Exxon Necton-37™ (FN 1380) and Exxon Mineral Seal Oil™ (FN 3200).

In an embodiment, the lubricating composition can be employed to lubricate a mechanical device. The mechanical device can be associated with an automotive vehicle. For example, the mechanical device may be a driveline device.

Driveline devices include automatic transmissions, manual transmission, dual clutch transmissions, or an axle or differential. A driveline device lubricating composition in different embodiments may have a composition as disclosed in the following table:

| | Embodiments (wt %) | | | |
|---|---|---|---|---|
| Additive | A² | B³ | C⁴ | D⁵ |
| Alkylbenzene sulfonate | 0.01 to 2 | 0.01 to 2 | 0.01 to 2 | 0.01 to 2 |
| detergent | | | | |
| Dispersant | 1 to 4 | 0.1 to 10, | 0 to 5 | 1 to 6 |
| | | 2 to 7 | | |
| Extreme Pressure Agent | 3 to 6 | 0 to 6 | 0 to 3 | 0 to 6 |
| Overbased Detergent | 0 to 1 | 0.01 to 3, | 0.5 to 6 | 0.01 to 2 |
| | | 0.025 to 2 | | |
| Antioxidant | 0 to 5 | 0.01 to 10 | 0 to 3 | 0 to 2 |
| | | or 3 | | |
| Friction Modifier | 0 to 5 | 0.01 to 5 | 0.1 to 1.5 | 0 to 5 |
| Viscosity Modifier¹ | 0.1 to 70 | 0.1 to 15 | 1 to 60 | 0.1 to 70 |
| Any Other Performance | 0 to 10 | 0 to 8 or | 0 to 6 | 0 to 10 |
| Additive | | 10 | | |

| | |
|---|---|
| Oil of Lubricating | Balance to 100% |
| Viscosity | |

Footnote:
¹The viscosity modifier in the table above may also be considered as an alternative to an oil of lubricating viscosity.
²Column A may be representative of an automotive or axle gear lubricant.
³Column B may be representative of an automatic transmission lubricant.
⁴Column C may be representative of an off-highway lubricant.
⁵Column D may be representative of a manual transmission lubricant.

The mechanical device lubricated with the lubricating composition disclosed herein can be an internal combustion engine, such as, for example, a spark ignited internal combustion engine or a compression ignition internal combustion engine. In one embodiment, the internal combustion engine may be one or more of a direct injection gasoline (GDI) engine or may be equipped with a turbocharger or supercharger. Gasoline engines may be operated at low speed, i.e. less than 3000 rpm, and at high pressure, i.e., greater than 12 bar brake mean effective pressure (BMEP). An engine lubricant composition in different embodiments may have a composition as disclosed in the following table:

| | Embodiments (wt %) | | |
|---|---|---|---|
| Additive | A | B | C |
| Alkylbenzene Sulfonate | 0.05 to 10 | 0.2 to 5 | 0.5 to 2 |
| detergent | | | |
| Other Overbased Detergent | 0 to 9 | 0.5 to 4 | 1 to 3 |
| Dispersant | 0 to 12 | 0.5 to 8 | 1 to 5 |
| Antioxidant | 0.1 to 13 | 0.3 to 8 | 0.5 to 5 |
| Antiwear Agent | 0.1 to 15 | 0.1 to 10 | 0.3 to 5 |
| Friction Modifier | 0.01 to 4 | 0.05 to 3 | 0.1 to 2 |
| Corrosion Inhibitor | 0 to 2 | 0.05 to 1 | 0.1 to 0.5 |
| Dispersant Viscosity | 0 to 5 | 0 to 4 | 0.05 to 2 |
| Modifier | | | |
| Viscosity Modifier | 0 to 10 | 0.5 to 8 | 1 to 6 |
| Any Other Performance | 0 to 10 | 0 to 8 | 0 to 6 |
| Additive | | | |

| | |
|---|---|
| Oil of Lubricating | Balance to 100% |
| Viscosity | |

The mechanical device may also be in a hydraulic system. A hydraulic lubricant may also comprise a formulation defined in the following table:

Hydraulic Lubricant compositions

| | Embodiments (wt %) | | |
|---|---|---|---|
| Additive | A | B | C |
| Alkylbenzene Sulfonate | 0.01 to 2.0 | 0.01 to 1.5 | 0.01 to 1.0 |
| detergent as described | | | |
| herein | | | |
| Antioxidant | 0 to 4.0 | 0.02 to 3.0 | 0.03 to 1.5 |
| Dispersant | 0 to 2.0 | 0.005 to 1.5 | 0.01 to 1.0 |
| Other Detergent - beside | 0 to 5.0 | 0.001 to 1.5 | 0.005 to 1.0 |
| alkyl benzene sulfonate | | | |
| detergent as described | | | |
| herein | | | |
| Anti-wear Agent | 0 to 5.0 | 0.001 to 2 | 0.1 to 1.0 |
| Friction Modifier | 0 to 3.0 | 0.02 to 2 | 0.05 to 1.0 |
| Viscosity Modifier | 0 to 10.0 | 0.5 to 8.0 | 1.0 to 6.0 |
| Any Other Performance | 0 to 1.3 | 0.00075 to 0.5 | 0.001 to 0.4 |
| Additive (antifoam/ | | | |
| demulsifier/pour point | | | |
| depressant) | | | |
| Metal Deactivator | 0 to 0.1 | 0.01 to 0.04 | 0.015 to 0.03 |
| Rust Inhibitor | 0 to 0.2 | 0.03 to 0.15 | 0.04 to 0.12 |
| Extreme Pressure Agent | 0 to 3.0 | 0.005 to 2 | 0.01 to 1.0 |
| Oil of Lubricating | Balance to | Balance to | Balance to |
| Viscosity | 100% | 100% | 100% |

The disclosed technology further provides a method of lubricating a circulating oil system.

The disclosed technology further provides a method of lubricating a turbine system.

The alkylbenzene sulfonate detergent described herein can be employed to lubricate any of the foregoing mechanical devices and/or internal combustion engine by supplying to the mechanical device and/or internal combustion engine with the aforementioned lubricating compositions.

### EXAMPLES

The invention will be further illustrated by the following examples, which set forth particularly advantageous embodiments. While the examples are provided to illustrate the invention, they are not intended to limit it.

A series of alkylbenzene sulfonate detergents according to aspects of the invention may be prepared by standard methods. Examples of these materials are represented by the formula

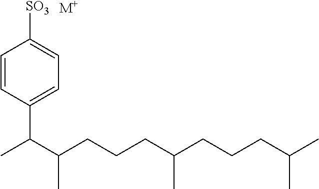

and summarized in Table 1.

TABLE 1
Alkylbenzene Sulfonate Detergents

| Additive | Metal (M) | Overbased | Metal Ratio | TBN (mg KOH/g) |
|---|---|---|---|---|
| ADD A | Calcium | — | 1.2 | 25 |
| ADD B | Calcium | Carbonate | 2.7 | 160 |
| ADD C | Calcium | Carbonate | 16 | 650 |
| ADD D | Magnesium | Carbonate | 14 | 660 |
| ADD E | (n-Butyl)₄N | Hydroxide | 3 | 100 |
| ADD F | Calcium | Carbonate | 10 | 480 |

A series of 5W-20 engine lubricants in Group II and Group III base oils of lubricating viscosity are prepared containing the detergent additives described above as well as conventional additives including polymeric viscosity modifier, other overbased detergents, antioxidants (combination of phenolic ester and diarylamine), zinc dialkyldithiophosphate (ZDDP), as well as other performance additives as follows (Table 2). The calcium, magnesium, phosphorus, zinc and ash contents of each of the examples are also presented in the table in part to show that each example has a similar amount of these materials and so provide a proper comparison between the comparative and invention examples.

TABLE 2
Engine Oil Lubricating Compositions¹

| | EX1 | EX2 | EX3 | EX4 | EX5 |
|---|---|---|---|---|---|
| Group III Base Oil | BALANCE to 100% | | | | |
| ADD A | | | | 0.3 | |
| ADD B | | | 0.4 | | |
| ADD C | | 0.85 | 0.6 | | 0.55 |
| ADD D | | | | 0.80 | |
| Overbased Calcium | 0.85 | | | | 0.3 |
| sulfonate² | | | | | |
| PIBsuccinimide | 2.7 | 2.7 | 2.7 | 2.7 | 2.7 |
| dispersant³ | | | | | |
| C3/C6 Secondary ZDDP | 0.78 | 0.78 | 0.78 | 0.78 | 0.78 |
| Ashless Antioxidant⁴ | 1.45 | 1.45 | 1.45 | 1.45 | 1.45 |
| Sulfurized olefin | 0.15 | 0.15 | 0.15 | 0.15 | 0.15 |
| Other Additives⁵ | 0.68 | 0.68 | 0.68 | 0.68 | 0.68 |
| Ethylene-propylene | 0.4 | 0.4 | 0.4 | 0.4 | 0.4 |
| VI improver | | | | | |
| CALCIUM (ppm) | 2260 | 2200 | 2120 | 150 | 2260 |
| MAGNESIUM (ppm) | | | | 1220 | |
| PHOSPHORUS (ppm) | 760 | 760 | 760 | 760 | 760 |
| ZINC (PPM) | 910 | 910 | 910 | 910 | 910 |

¹All treat rates are oil free unless otherwise indicated
²500 TBN overbased calcium alkylbenzene sulfonates
³Conventional polyisobutenylsuccinimide dispersant (TBN 54 mm KOH/g)
⁴Combination of alkylated diphenylamine and hindered phenol ester antioxidants
⁵Other additives include corrosion inhibitors, pourpoint depressants, ashless friction modifiers, and foam inhibitors

### Evaluation of Engine Lubricant Compositions

Engine lubricant formulations may be subject to bench and engine tests designed to evaluate the ability of the lubricant, and thus the detergent, to prevent deposit formation, provide cleanliness, reduce or prevent acid-mediated wear or degradation of the lubricant, and provide sludge handling. Engine tests include the Caterpillar 1K (ASTM D6750) single cylinder engine test, the Volkswagen TDI test (VW TDI CEC-L-78-T-99 test, also known as the PV1452) which measures the deposit control performance of engine lubricants, the API SN sludge test (Sequence VG), and the MB M271 SL Sludge Engine Test, which test is an industry standard for evaluating a lubricating composition to mitigate sludge in internal combustion engines.

Suitable bench tests include the MHT TEOST (ASTM D7097B); the TEOST 33C (ASTM D6335); hot tube testing (e.g. Komatsu Hot Tube or KHT) wherein the oil sample is contacted with a glass tube at high temperature (typically 280° C.) for a period of time (typically 16 hours), rinsed, and the cleanliness of the tube is rated; and panel coker testing wherein the oil sample is splashed onto a metal panel held at 325° C. in a cycle of splashing and baking for 3.5 hours, and the panel is weighed to determine amount of deposit formation and a visible rating is carried out.

A series of driveline lubricants are prepared containing the detergent additives. Examples 6, 7, and 8 are fluids for use in an automatic transmission, a farm tractor and a manual transmission respectively. These fluid compositions are prepared according to the recipes listed in Table 3.

TABLE 3
Driveline Lubricating Compositions

| | EX6 | EX7 | EX8 |
|---|---|---|---|
| ADD A | 0.15 | | |
| ADD C | | | 0.15 |
| ADD F | 0.09 | 0.9 | |
| Overbased calcium | | 1.6 | |
| detergent | | | |
| Substituted thiadizole | 0.08 | | 0.2 |
| dispersant | | 0.25 | |
| Borated dispersant | 3.1 | | 0.86 |
| Friction modifier | 1.39 | 0.5 | |
| Dialkyl phosphite | 0.2 | | 0.2 |
| Aminic Antioxidant | 0.66 | | 0.3 |
| Antiwear | 0.53 | 1.9 | 0.75 |
| Seal swell | 1.1 | | |
| Oil | 1.3 | 0.2 | 1.8 |
| VM | 4.9 | 1.8 | 5.7 |
| Base oil | 86.5 | 92.8 | 90 |
| CALCIUM (ppm) | 256 | 3600 | 390 |
| PHOSPHORUS (ppm) | 652 | 1200 | 310 |
| ZINC (ppm) | | 1494 | |

Friction and wear performance in driveline devices can be measured using a variety of tests, often depending on the final end use of the fluid. The FZG test measures the anti-scuffing properties of oil for reduction gears, hypoid gears, automatic transmission gears and the like. A description of the FZG test and the meaning of the results is found in the article “Scuffing Tests on Gear Oils in the FZG Apparatus” by G. Niemann, H. Rettig and G. Lechner in ASLE Transactions, 4 71-86 (1961). Test procedure DIN 51354 is utilized which is discussed in Prufung von Schmierstoffen: Mechanische Prufung von Gebriebeolen in der FZG-Zahnrad-Verspannungs-Prufmaschine, January 1970. The results reported include load stage failure. Typically, better results are obtained for lubricants reporting a higher load stage failure.

Friction performance may be measured using the VSFT test procedure, the SAE#2 and/or a synchronizer test rig in a “durability test”. The VSFT test procedure consists of a disc that can be metal or another friction material which is rotated against a metal surface. The friction materials employed in the particular tests are various commercial friction materials commonly used in automatic transmission clutches. The test is run over three temperatures and two load levels. The coefficient of friction measured by the VSFT is plotted against the sliding speed over several speed sweeps at a constant pressure.

The amount of each chemical component described is presented exclusive of any solvent or diluent oil, which may be customarily present in the commercial material, that is, on an active chemical basis, unless otherwise indicated. However, unless otherwise indicated, each chemical or composition referred to herein should be interpreted as being a commercial grade material which may contain the isomers, by-products, derivatives, and other such materials which are normally understood to be present in the commercial grade.

It is known that some of the materials described above may interact in the final formulation, so that the components of the final formulation may be different from those that are initially added. For instance, metal ions (of, e.g., a detergent) can migrate to other acidic or anionic sites of other molecules. The products formed thereby, including the products formed upon employing the composition of the present invention in its intended use, may not be susceptible of easy description. Nevertheless, all such modifications and reaction products are included within the scope of the present invention; the present invention encompasses the composition prepared by admixing the components described above.

As used herein, the term “about” means that a value of a given quantity is within ±20% of the stated value. In other embodiments, the value is within ±15% of the stated value. In other embodiments, the value is within ±10% of the stated value. In other embodiments, the value is within ±5% of the stated value. In other embodiments, the value is within ±2.5% of the stated value. In other embodiments, the value is within ±1% of the stated value.

Additionally, as used herein, the term “substantially” means that a value of a given quantity is within ±10% of the stated value. In other embodiments, the value is within ±5% of the stated value. In other embodiments, the value is within ±2.5% of the stated value. In other embodiments, the value is within ±1% of the stated value.

Each of the documents referred to above is incorporated herein by reference, including any prior applications, whether or not specifically listed above, from which priority is claimed. The mention of any document is not an admission that such document qualifies as prior art or constitutes the general knowledge of the skilled person in any jurisdiction. Except in the Examples, or where otherwise explicitly indicated, all numerical quantities in this description specifying amounts of materials, reaction conditions, molecular weights, number of carbon atoms, and the like, are to be understood as modified by the word “about.” It is to be understood that the upper and lower amount, range, and ratio limits set forth herein may be independently combined. Similarly, the ranges and amounts for each element of the invention can be used together with ranges or amounts for any of the other elements.

As used herein, the transitional term “comprising,” which is synonymous with “including,” “containing,” or “characterized by,” is inclusive or open-ended and does not exclude additional, un-recited elements or method steps. However, in each recitation of “comprising” herein, it is intended that the term also encompass, as alternative embodiments, the phrases “consisting essentially of” and “consisting of,” where “consisting of” excludes any element or step not specified and “consisting essentially of” permits the inclusion of additional un-recited elements or steps that do not materially affect the essential or basic and novel characteristics of the composition or method under consideration.

While certain representative embodiments and details have been shown for the purpose of illustrating the subject invention, it will be apparent to those skilled in this art that various changes and modifications can be made therein without departing from the scope of the subject invention. In this regard, the scope of the invention is to be limited only by the following claims.

What is claimed is:

1. An alkylbenzene sulfonate detergent composition comprising:
at least one benzene sulfonate moiety having a hydrocarbyl group attached thereto, the hydrocarbyl group including at least one oligomer having monomers equivalent to 5 to 10 carbon atom branched olefins.

2. The alkylbenzene sulfonate detergent composition of claim 1, where the monomers are equivalent to 5 to 10 carbon atom branched polyenes.

3. The alkylbenzene sulfonate detergent composition of claim 2, where the monomers are equivalent to isoprene.

4. The alkylbenzene sulfonate detergent composition of any one of the preceding claims, where the oligomer is a terpene.

5. The alkylbenzene sulfonate detergent composition of any one of the preceding claims, where the oligomer is equivalent to at least one of formula:

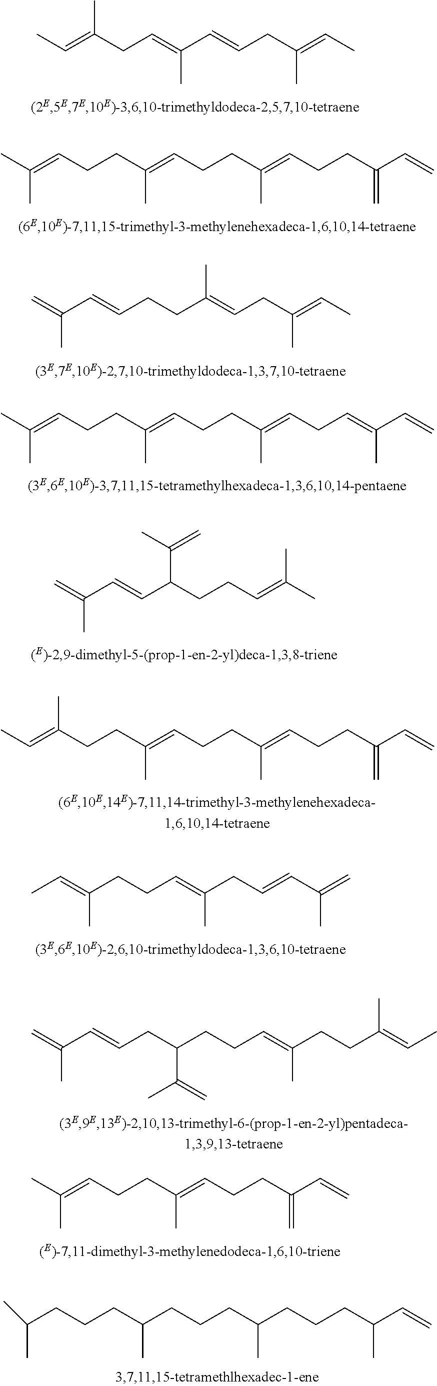

6. The alkylbenzene sulfonate detergent composition of any one of the preceding claims, wherein the oligomer is equivalent to a partially or completely hydrogenated form of a terpene.

7. The alkylbenzene sulfonate detergent composition of claim 5, wherein the oligomer is equivalent to any of formula:

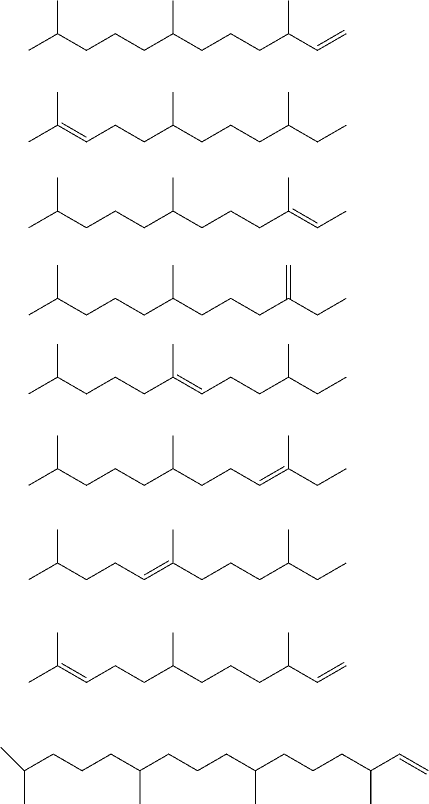

8. The alkylbenzene sulfonate detergent composition of any one of the preceding claims, wherein the hydrocarbyl group has 15 to 60 carbon atoms.

9. The alkylbenzene sulfonate detergent composition of any one of the preceding claims, wherein the detergent comprises any one of formula: 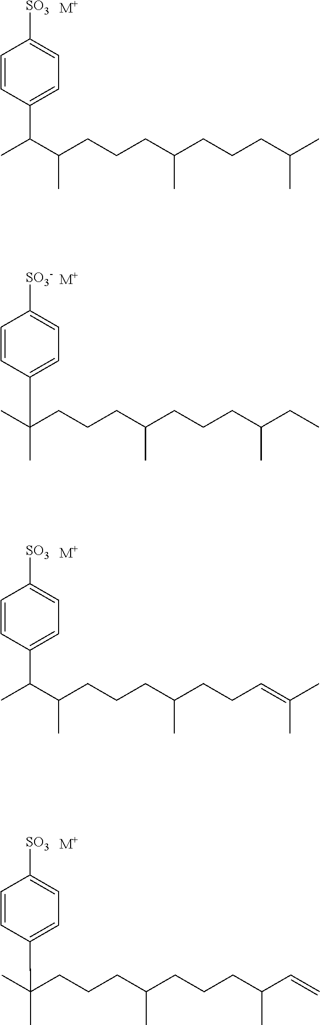
wherein,
M is selected from an alkali metal, an alkaline earth metal, a Group 3 through Group 12 transition metal, main group metal, and a quaternary ammonium compound represented by NR₄⁺X⁻, where each R independently is a hydrocarbyl group.

10. The alkylbenzene sulfonate detergent composition of any one of the preceding claims, wherein the detergent comprises one or more alkali metals, one or more alkaline earth metals, or mixtures thereof.

11. The alkylbenzene sulfonate detergent composition of any one of the preceding claims, wherein the alkaline earth metal is selected from magnesium and calcium.

12. The alkylbenzene sulfonate detergent composition of any one of the preceding claims, wherein the detergent is overbased.

13. The alkylbenzene sulfonate detergent composition of claim 12, wherein the overbased detergent has a metal ratio of at least 1.5, at least 5, or at least 7.

14. The alkylbenzene sulfonate detergent composition of any one of claims 1 to 11, wherein the detergent is neutral.

15. The alkylbenzene sulfonate detergent composition of any of the preceding claims, wherein the alkylbenzene sulfonate detergent has a TBN of at least 400.

16. A lubricating composition comprising
(a) an oil of lubricating viscosity; and
(b) the alkylbenzene sulfonate detergent composition of any one of the preceding claims.

17. The lubricating composition of claim 16, wherein the alkylbenzene sulfonate detergent is present from about 0.01 to about 2 wt %, or from about 0.1 to about 1.75 wt %, or about 0.2 to about 1.5 wt % of the lubricating composition.

18. The Lubricating composition of any one of claims 16 to 17, further comprising an antiwear agent.

19. The lubricating composition of claim 18, wherein the antiwear agent is zinc dialkyldithiophosphate.

20. The lubricating composition of any one of claims 18 to 19, wherein the antiwear agent is present in the lubricating composition in an amount of from 0.1 to 15 wt %, or 0.1 to 10 wt % or 0.3 to 5 wt %, or 0 to 5.0 wt %, or 0.001 to 2 wt %, or 0.1 to 1.0 wt %.

21. The lubricating composition of any one of claims 16 to 20, further comprising a dispersant.

22. The lubricating composition of claim 21, wherein the dispersant is present at 0.1 to 5 wt %, or 0.3 to 4 wt %, or 0.5 to 3 wt %, or 1 to 2.5 or 3 wt %, or 1.5 to 4 wt %, or 1.5 to 3 wt %, or even 0.1 to 3 wt %, or 0.1 to 2.5 wt %, or 0.2 to 2 wt %.

23. The lubricating composition of any one of claims 21 to 22, wherein the dispersant is a polyisobutylene succinimide dispersant.

24. The lubricating composition of any one of claims 16 to 23, further comprising a phosphorus-containing antiwear agent chosen from (i) a non-ionic phosphorus compound, which may be a hydrocarbyl phosphite; or (ii) an amine salt of a phosphorus compound.

25. A method of lubricating a mechanical device comprising supplying to the device the lubricating composition of any one of claims 16 to 24.

26. The method of claim 25, wherein the mechanical device is selected from a driveline device, an internal combustion engine, and a hydraulic system.

27. The method of claim 25, wherein the internal combustion engine is selected from a heavy-duty diesel compression ignited internal combustion engine, and a spark assisted compression ignited internal combustion engine.

28. The method of claim 26, wherein the internal combustion engine is a direct injection gasoline engine equipped with a turbocharger operated at a pressure of greater than 12 bar brake mean effective pressure at less than 3,000 rpm.

29. The method of claim 26, wherein the mechanical device is an automatic transmission.

30. The method of claim 26, wherein the mechanical device is a manual transmission.

31. The method of claim 26, wherein the mechanical device is an automotive axle.

32. The method of claim 26, wherein the mechanical device is an off-highway vehicle.

33. The method of any one of the preceding claims, wherein the hydrocarbyl group of the alkylbenzene sulfonate detergent comprises at least 25 wt %, or at least 50 wt %, or at least 75 wt %, or at least 80 wt % or at least 90 wt %, or at least 95 wt %, or 100 wt % of biorenewable content.
